SEQUENCE LISTING (1 sequence)

SEQ ID NO: 1 - protein, 14 aa, Artificial
  Synthetic 1W1K peptide
EAWGALANKAVDKA

(19) United States
(12) Patent Application Publication
(10) Pub. No.: US 2016/0193328 A1
(43) Pub. Date: Jul. 7, 2016

(54) Immunoadjuvant Compositions and uses Thereof

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ PAUL SABATIER TOULOUSE III, Toulouse (FR)

(72) Inventors: Nicolas Fazilleau, Toulouse (FR); Svetoslav Chakarov, Toulouse (FR)

(21) Appl. No.: 14/911,944

(22) PCT Filed: Aug. 14, 2014
(86) PCT No.: PCT/EP2014/067415

§ 371 Date: Feb. 12, 2016

(30) Foreign Application Priority Data
Aug. 14, 2013 (EP) 13306150.7

(51) Int. Cl. A61K 39/39

(52) U.S. Cl. CPC A61K 39/39; A61K 2039/575

(57) ABSTRACT

The present invention relates to an immunoadjuvant composition comprising at least adjuvant and at least one MyD88-dependent pathway agonist.

### FIELD OF THE INVENTION

The present invention relates to an immunoadjuvant composition comprising at least one adjuvant and at least one MyD88-dependent pathway agonist.

### BACKGROUND OF THE INVENTION

Most vaccines are designed empirically using attenuated pathogens as the source of foreign antigens (Ag). Improving vaccine efficacy has, however, proven difficult, mainly, because the fundamental immune mechanisms of vaccine action remain elusive. Toll-like receptors (TLR) are innate immune receptors that specifically recognize pathogen challenges and are pivotal for initiating inflammation, but have also emerged as one of the most important families of receptors for the priming of adaptive immune responses [Banchereau and Steinman, 1998]. TLR ligands are thus widely used as vaccine adjuvants in different clinical settings.

The intra-cellular adapter molecule Myeloid differentiation factor 88 (MyD88) is required for the transduction of signals from all TLR except TLR3, and thus for the adjuvanticity of TLR ligands such as CpG oligonucleotides (ODN) used as TLR9 agonist or lipopolysaccharide (LPS) as TLR4 agonist. In contrast, Toll interleukin 1 receptor domain-containing adapter inducing interferon beta (TRIF) associates with TLR3 and TLR4 and signals, for example, TLR3 recognition of its agonist poly(I:C). TLR9, which is localized to the endosomal membrane of B cells, plasmacytoid dendritic cells (pDC) and to a lesser extent dendritic cells (DC), is a sensor for DNA enriched in hypomethylated CpG sequences, such as found in DNA of bacterial or viral origin [Klinman, 2004]. Upon recognition of CpG by TLR9, MyD88 induces phosphorylation of interferon regulatory factor 7 (IRF-7), which is required for transcriptional activity of type I interferon (IFN). MyD88 engagement can also lead to the activation of nuclear factor-κB (NF-κB), which in turn induces transcription of pro-inflammatory cytokines such as IL-6 and TNF-α [Klinman, 2004]. Moreover, depending on the physical context in which CpG is presented, MyD88 signalling can substantially enhance immunoglobulin (Ig) responses to protein Ag. Using a mouse model that lacks MyD88 selectively in CD11c+DC or in B cells, soluble CpG monotherapy has been shown to increase the in vivo T-dependent Ab response to protein Ag selectively through CD11c+DC, which is expressed in both pDC and DC [Hou et al., 2011]. In contrast, the adjuvant activity of CpG on Ab response is dependent on MyD88 expression in B cells when CpG is delivered in virus like particle [Hou et al., 2011]. TLR4 is the receptor for Gram-negative LPS and monophosphoryl lipid A (MPL), its toxic moiety. TLR4 is expressed at the surface of monocytes and at very low levels on human B cells. Although they are both recognized by TLR4, MPL signals mainly via TRIF while LPS signals via both adapters, MyD88 and TRIF [Mata-Haro et al., 2007]. MPL is licensed for use as a vaccine adjuvant and was shown to promote neutralizing Ab and memory B cell formation in vaccine setting [Pulendran and Ahmed, 2011].

Protein vaccines can promote long-term immunity through the differentiation of Ag-specific high-affinity memory B cells and long-lived plasma cells (PC). To be effective, vaccine priming must induce Ag-specific helper T cells that are required to regulate the emerging B cell response. It is now clear that this cognate T cell help involves a distinct lineage of CD4+ T cells named T Follicular Helper cells (Tfh) [Crotty S., 2011]. Tfh cells control PC production and memory B cell development in secondary lymphoid tissues through a combination of specific TCR-peptide-MHCII (pMHCII) interactions, engagement of costimulatory molecules and cytokine delivery. Several recent studies have demonstrated that the master transcriptional regulator Bcl-6 drives Tfh cell differentiation. The inventors have also shown that the strength of Ag-specific TCR binding directly influences the differentiation of Tfh cells in vivo [Fazilleau N. et al., 2009]. Bcl-6 induces the expression of the chemokine receptor CXCR5, which is the hallmark of Tfh cells. CXCR5 promotes Tfh cell migration in CXCL13-rich areas such as the T-B border and the B follicles, where they regulate the outcome of the Ag-specific B cell response. Before germinal centre (GC) formation, Tfh cells most likely provide help either at the T-B border or in interfollicular zones. Tfh cells direct Ag-primed B cells into the short-lived PC pathway. Alternatively, some primed B cells associated with Tfh cells move towards the B cell zones to form the GC reaction where B cell receptor diversification processes occur via somatic hypermutation. Tfh cells in the GC are involved in the control of B cell maturation by improving antibody (Ab) affinity maturation and preserving self-tolerance. The GC reaction produces two categories of affinity-matured memory B cells. On the one hand, the most typical memory B cells are the precursors for a memory response to Ag recall, and do not initially secrete Ab. On the other hand, the long-lived PC are terminally differentiated cells that continually produce and secrete high-affinity Ab and are not drawn into a secondary response.

It has been shown that the Tfh genetic program is driven in vivo by IL-6 and IL-21 and occurs preferentially in lymphoid organs draining the site of immunisation [Fazilleau et al., 2007]. Additionally, while Tfh cells control B cell maturation, interactions with B cells are reciprocally essential for Tfh differentiation. In both B cell-deficient mice and transgenic (tg) mice harbouring a B cell repertoire with Ag specificity different from that of the T cell compartment, there is an impaired Tfh cell development. Moreover, ICOS-L expression at the surface of B cells has been shown to be important for Tfh cell differentiation in vivo, through its interaction with ICOS. Deficiency in SLAM-associated protein (SAP) selectively impairs the ability of CD4+ T cells to stably interact with cognate B cells but not with DC, leading to a defective CD4+ T cell recruitment and retention within GC as differentiated Tfh cells. Finally, after immunisation with protein Ag in adjuvant, type I IFN secreted by pDC induces a signalling cascade in conventional CD11c+ DC (cDC) that can lead to the induction of Tfh cell differentiation [Cucak et al., 2009].

The use of TCR tg models implies non-physiological frequency of naive Ag-specific CD4+ T cells, which can impact the outcome of an immune response, via mechanisms such as clonal expansion and/or development of T cell memory in vivo (Badovinac et al., 2007; Ford et al., 2007; Hataye et al., 2006; Marzo et al., 2005). Hence, for the study of the inventors, they used three different Ag models for which they have direct access to polyclonal and endogenous Ag-specific CD4+ T cells, Ag-specific B cells and Ag-presenting DC in non-tg wild-type (wt) mice following immunisation. Adjuvant combinations have been proposed to possibly result in synergistic enhancement of immune response to vaccine and adjuvanticity. Therefore, how addition of soluble TLR agonists to classical adjuvant contributes to Tfh cell differentiation and Ab responses in vivo remained poorly characterized to date.

### SUMMARY OF THE INVENTION

Here, the inventors show that addition to classical adjuvants of soluble MyD88-dependent TLR agonists such as ODN containing an unmethylated CpG motif or LPS enhances Ag-specific Tfh cells in vivo without changing the overall extent of the Ag-specific CD4+ T cell response. In contrast, the TLR agonist poly(I:C) has no enhancing effect on Ag-specific Tfh cells. The Tfh cell promotion correlates with an enhancement of Ag-specific GC B cells, PC and seric Ig. Furthermore, they show that Ag-presenting CD11b+ monocyte-derived DC (moDC) are responsible for Tfh cell increase as shown in vivo in their absence. These latter mediate this phenomenon through secretion of IL-6. In contrast, they also document that neither B cells nor pDC take a detectable part in this bias towards Tfh cell differentiation while they also secrete IL-6 in response to CpG. Overall, these results suggest that some TLR ligands appear to imprint the specialized program of Ag-specific effector Tfh function needed to promote high-affinity B cell immunity in vivo specifically through moDC.

Thus, the invention relates to an immunoadjuvant composition comprising at least one adjuvant and at least one MyD88-dependent pathway agonist.

### DETAILED DESCRIPTION OF THE INVENTION

A first aspect of the invention relates an immunoadjuvant composition comprising:
a. at least one adjuvant and;
b. at least one MyD88-dependent pathway agonist.

As used herein, the term “immunoadjuvant composition” refers to a composition that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “immunoadjuvant composition” means a composition that increases the differentiation of antigen-specific T Follicular helper cells (Tfh). This phenomenon correlated with an enhancement of germinal centre reaction, antigen-specific plasma cells and circulating antibodies. Specifically, the “immunoadjuvant composition” is responsible for the activation of the CD11b⁺ monocyte-derived DC that produced IL-6 that also enhances the development of the Tfh compartment. In response to this mechanism, the humoral response (production of antibody against a specific antigen) is improved.

Thus, the invention also relates to an immunoadjuvant composition comprising:
a. at least one adjuvant and;
b. at least one MyD88-dependent pathway agonist;

for use in the improvement of the humoral response in a subject in need thereof.

As used herein, the term “adjuvant” refers to a substance that enhances, augments or potentiates the host's immune response to an antigen, e.g., an antigen that is part of a vaccine. Non-limiting examples of some commonly used vaccine adjuvants include insoluble aluminum compounds, calcium phosphate, liposomes, Virosomes™, ISCOMS®, microparticles (e.g., PLG), emulsions (e.g., MF59, Montanides), virus-like particles & viral vectors. PolyICLC (a synthetic complex of carboxymethylcellulose, polyinosinic-polycytidylic acid, and poly-L-lysine double-stranded RNA), which is a TLR3 agonist, is used as an adjuvant in the present invention. It will be understood that other TLR agonists may also be used (e.g. TLR4 agonists, TLR5 agonists, TLR7 agonists, TLR9 agonists), or any combinations or modifications thereof.

Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE, MF59 and RIBI (also known as SAS), which contains Monophosphoryl Lipid A (MPL)(detoxified endotoxin) from Salmonella minnesota and synthetic Trehalose Dicorynomycolate (TDM) in 2% oil (squalene)-Tween® 80-water (see for review Pulendran and Ahmed, 2011). Other examples of adjuvants include DDA (dimethyldioctadecylammonium bromide), Freund's complete and incomplete adjuvants and QuilA. In addition, immune modulating substances such as lymphokines (e.g., IFN-[gamma], IL-2 and IL-12) or synthetic IFN-[gamma] inducers such as poly I:C can be used in combination with adjuvants described herein.

In one embodiment, the adjuvant may be a “MPL based adjuvant” like MF59 or RIBI (SAS) that is to say an adjuvant with MPL with one or several excipients in oil, water or mixture thereof solution.

Suitable adjuvants include any acceptable immunostimulatory compound, such as cytokines, chemokines, cofactors, toxins, plasmodia, synthetic compositions or vectors encoding such adjuvants.

Adjuvants that may be used in accordance with embodiments include, but are not limited to, IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains Monophosphoryl Lipid A (MPL)(detoxified endotoxin) from Salmonella minnesota and synthetic Trehalose Dicorynomycolate (TDM) in 2% oil (squalene)-Tween® 80-water is also contemplated. MHC antigens may even be used. Exemplary adjuvants may include complete Freund's adjuvant (a non-specific stimulator of the immune response containing killed Mycobacterium tuberculosis), incomplete Freund's adjuvants and/or aluminum hydroxide adjuvant.

In one embodiment, the immunoadjuvant composition according to the invention comprises and adjuvant selected from the group consisting of Freund's complete (IFA), Alum, squalene and RIBI (also called thereafter SAS).

As used herein, the term “MyD88-dependent pathway” denotes a pathway which implies the Myeloid differentiation factor 88 (MyD88) for the transduction of signals from all TLR except TLR3.

In one embodiment the MyD88-dependent pathway agonist according to the invention is selected from the group consisting of TLR4 and TLR9 agonists and mixture thereof (see for example Janeway C A et al. 2002).

As used herein, the term “TLR4 agonist” denotes a compound or a molecule that binds the Toll-like receptor 4 and active it. According to the invention, a TLR4 agonist may be selected from the group consisting of Ethanol, Morphine-3-glucuronide, Morphine, Oxycodone, Levorphanol, Pethidine, Glucuronoxylomannan from Cryptococcus, Fentanyl, Methadone, Buprenorphine, Lipopolysaccharides (LPS), Carbamazepine, Oxcarbazepine.

In a particular embodiment, the TLR4 agonist according to the invention is selected from the group consisting of the LPS.

Various TLR4 agonists are known in the art, including Monophosphoryl lipid A (MPLA), in the field also abbreviated to MPL, referring to naturally occurring components of bacterial lipopolysaccharide (LPS); refined detoxified endotoxin. For example, MPL is a derivative of lipid A from Salmonella minnesota R595 lipopolysaccharide (LPS or endotoxin). While LPS is a complex heterogeneous molecule, its lipid A portion is relatively similar across a wide variety of pathogenic strains of bacteria. MPL, used extensively as a vaccine adjuvant, has been shown to activate TLR4 (Martin M. et al., 2003. Infect Immun. 71(5):2498-507; Ogawa T. et al., 2002. Int Immunol. 14(11):1325-32). TLR4 agonists also include natural and synthetic derivatives of MPLA, such as 3-de-O-acylated monophosphoryl lipid A (3D-MPL), and MPLA adjuvants available from Corixa Corporation (Seattle, Wash.; see U.S. Pat. Nos. 4,436,727; 4,436,728; 4,987,237; 4,877,611; 4,866,034 and 4,912,094 for structures and methods of isolation and synthesis). A structure of MPLA is disclosed in U.S. Pat. No. 4,987,237. Non-toxic diphosphoryl lipid A (DPLA) may also be used, for example OM-174, a lipid A analogue of bacterial origin containing a triacyl motif linked to a diglucosamine diphosphate backbone. Another class of useful compounds are synthetic lipid A analogue pseudo-dipeptides derived from amino acids linked to three fatty acid chains (see for example EP 1242365), such as OM-197-MP-AC, a water soluble synthetic acylated pseudo-dipeptide (C55H107N4O12P). Non-toxic TLR4 agonists include also those disclosed in EP1091928, PCT/FR05/00575 or PCT/IB2006/050748. PCT/US2006/002906/WO 2006/083706; PCT/US 2006/003285/WO 2006/083792; PCT/US 2006/041865; PCT/US 2006/042051. TLR4 agonists also include synthetic compounds which signal through TLR4 other than those based on the lipid A core structure, for example an aminoalkyl glucosaminide 4-phosphate (see Evans J T et al. Expert Rev Vaccines. 2003 April; 2(2):219-29; or Persing et al. Trends Microbiol. 2002; 10(10 Suppl):S32-7. Review). Other examples include those described in Orr M T, Duthie M S, Windish H P, Lucas E A, Guderian J A, Hudson T E, Shaverdian N, O'Donnell J, Desbien A L, Reed S G, Coler R N. MyD88 and TRIF synergistic interaction is required for TH1-cell polarization with a synthetic TLR4 agonist adjuvant. Eur J Immunol. 2013 May 29. doi: 10.1002/eji.201243124.; Lambert S L, Yang C F, Liu Z, Sweetwood R, Zhao J, Cheng L, Jin H, Woo J. Molecular and cellular response profiles induced by the TLR4 agonist-based adjuvant Glucopyranosyl Lipid A. PLoS One. 2012; 7(12):e51618. doi: 10.1371/journal.pone.0051618. Epub 2012 Dec. 28.

As used herein, the term “TLR9 agonist” denotes a compound or a molecule that binds the Toll-like receptor 9 and actives it (see for example Klinman D M 2004). According to the invention, a TLR9 agonist may be selected from the group consisting of CpG oligonucleotides (ODN) and its derivatives.

In particular embodiment, the TLR9 agonist is the CpG (ODN).

In another particular embodiment, the CpG can be a CpG A, B or C (Krieg A. 2002 or Klinman D M 2004).

In still another particular embodiment, the CpG is the CpG-B.

Examples of TLR9 agonists (include nucleic acids comprising the sequence 5′-CG-3′ (a “CpG nucleic acid”) in certain aspects C is unmethylated. The terms “polynucleotide,” and “nucleic acid,” as used interchangeably herein in the context of TLR9 agonist molecules, refer to a polynucleotide of any length, and encompasses, inter alia, single- and double-stranded oligonucleotides (including deoxyribonucleotides, ribonucleotides, or both), modified oligonucleotides, and oligonucleosides, alone or as part of a larger nucleic acid construct, or as part of a conjugate with a non-nucleic acid molecule such as a polypeptide. Thus a TLR9 agonist may be, for example, single-stranded DNA (ssDNA), double-stranded DNA (dsDNA), single-stranded RNA (ssRNA) or double-stranded RNA (dsRNA). TLR9 agonists also encompass crude, detoxified bacterial (e.g., mycobacterial) RNA or DNA, as well as enriched plasmids enriched for a TLR9 agonist. In some embodiments, a “TLR9 agonist-enriched plasmid” refers to a linear or circular plasmid that comprises or is engineered to comprise a greater number of CpG motifs than normally found in mammalian DNA. Examples of non-limiting TLR9 agonist-enriched plasmids are described in Roman et al. (1997). In general, a TLR9 agonist used in a subject composition comprises at least one unmethylated CpG motif. In some embodiments, a TLR9 agonist comprises a central palindromic core sequence comprising at least one CpG sequence, where the central palindromic core sequence contains a phosphodiester backbone, and where the central palindromic core sequence is flanked on one or both sides by phosphorothioate backbone-containing polyguanosine sequences. In other embodiments, a TLR9 agonist comprises one or more TCG sequences at or near the 5′ end of the nucleic acid; and at least two additional CG dinucleotides. In some of these embodiments, the at least two additional CG dinucleotides are spaced three nucleotides, two nucleotides, or one nucleotide apart. In some of these embodiments, the at least two additional CG dinucleotides are contiguous with one another. In some of these embodiments, the TLR9 agonist comprises (TCG)n, where n=1 to 3, at the 5′ end of the nucleic acid. In other embodiments, the TLR9 agonist comprises (TCG)n, where n=1 to 3, and where the (TCG)n sequence is flanked by one nucleotide, two nucleotides, three nucleotides, four nucleotides, or five nucleotides, on the 5′ end of the (TCG)n sequence. A TLR9 agonist of the present invention includes, but is not limited to, any of those described in U.S. Pat. Nos. 6,194,388; 6,207,646; 6,239,116; 6,339,068; and 6,406,705, 6,426,334 and 6,476,000, and published US Patent Applications US 2002/0086295, US 2003/0212028, and US 2004/0248837.

Examples of others TLR4 or TLR9 agonists are described in WO 2012/021834, the contents of which are incorporated herein by reference.

In another particular embodiment, the immunoadjuvant composition according to the invention comprises at least one adjuvant, at least one TLR4 agonist and at least one TLR9 agonist.

In another particular embodiment, the immunoadjuvant composition according to the invention comprises the adjuvant RIBI and the TLR9 agonist CpG (ODN).

In another particular embodiment, the immunoadjuvant composition according to the invention comprises the adjuvant RIBI and the TLR9 agonist CpG-B.

In another particular embodiment, the immunoadjuvant composition according to the invention comprises the adjuvant IFA, the TLR4 agonist LPS and the TLR9 agonist CpG (ODN).

In another particular embodiment, the immunoadjuvant composition according to the invention comprises the adjuvant Alum, the TLR4 agonist LPS and the TLR9 agonist CpG (ODN).

In another particular embodiment, the immunoadjuvant composition according to the invention comprises the adjuvant RIBI, the TLR4 agonist LPS and the TLR9 agonist CpG (ODN).

In another particular embodiment, the immunoadjuvant composition according to the invention comprises the adjuvant squalene, the TLR4 agonist LPS and the TLR9 agonist CpG (ODN).

In another particular embodiment, the immunoadjuvant composition according to the invention comprises the adjuvant IFA, the TLR4 agonist LPS and the TLR9 agonist CpG-B.

In another particular embodiment, the immunoadjuvant composition according to the invention comprises the adjuvant Alum, the TLR4 agonist LPS and the TLR9 agonist CpG-B.

In another particular embodiment, the immunoadjuvant composition according to the invention comprises the adjuvant squalene, the TLR4 agonist LPS and the TLR9 agonist CpG-B.

In another particular embodiment, the immunoadjuvant composition according to the invention comprises a MPL based adjuvant, the TLR4 agonist LPS and the TLR9 agonist CpG.

In another particular embodiment, the immunoadjuvant composition according to the invention comprises a MPL based adjuvant, the TLR4 agonist LPS and the TLR9 agonist CpG-B.

In another particular embodiment, the immunoadjuvant composition according to the invention comprises MPL, the TLR4 agonist LPS and the TLR9 agonist CpG.

In another particular embodiment, the immunoadjuvant composition according to the invention comprises MPL, the TLR4 agonist LPS and the TLR9 agonist CpG-B.

In another particular embodiment, the immunoadjuvant composition according to the invention comprises MF59, the TLR4 agonist LPS and the TLR9 agonist CpG.

In another particular embodiment, the immunoadjuvant composition according to the invention comprises MF59, the TLR4 agonist LPS and the TLR9 agonist CpG-B.

In another particular embodiment, the immunoadjuvant composition according to the invention comprises MF59, the TLR4 agonist LPS and the TLR9 agonist CpG.

In another particular embodiment, the immunoadjuvant composition according to the invention comprises MF59, the TLR4 agonist LPS and the TLR9 agonist CpG-B.

As used herein the term “antigen” refers to a molecule capable of being specifically bound by an antibody or by a T cell receptor (TCR) if processed and presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

A further object of the invention relates to a vaccine composition, comprising at least one antigen, at least one adjuvant (as above defined), at least one MyD88-dependent pathway agonist (as above defined) and optionally with one or more pharmaceutically acceptable excipients.

A “vaccine composition”, once it has been administered to a subject or an animal, elicits a protective immune response against said one or more antigen(s) that is (are) comprised herein. Accordingly, the vaccine composition of the invention, once it has been administered to the subject or the animal, induces a protective immune response against, for example, a microorganism, or to efficaciously protect the subject or the animal against infection.

A variety of substances can be used as antigens in a compound or formulation, of immunogenic or vaccine type. For example, attenuated and inactivated viral and bacterial pathogens, purified macromolecules, polysaccharides, toxoids, recombinant antigens, organisms containing a foreign gene from a pathogen, synthetic peptides, polynucleic acids, antibodies and tumor cells can be used to prepare (i) an immunogenic composition useful to induce an immune response in a individual or (ii) a vaccine useful for treating a pathological condition.

Therefore, the immunoadjuvant composition of the invention can be combined with a wide variety of antigens to produce a vaccine composition useful for inducing an immune response in an individual.

Those skilled in the art will be able to select an antigen appropriate for treating a particular pathological condition and will know how to determine whether an isolated antigen is favored in a particular vaccine formulation.

An isolated antigen can be prepared using a variety of methods well known in the art. A gene encoding any immunogenic polypeptide can be isolated and cloned, for example, in bacterial, yeast, insect, reptile or mammalian cells using recombinant methods well known in the art and described, for example in Sambrook et al., Molecular cloning: A Laboratory Manual, Cold Spring Harbor Laboratory, New York (1992) and in Ansubel et al., Current Protocols in Molecular Biology, John Wiley and Sons, Baltimore, Md. (1998). A number of genes encoding surface antigens from viral, bacterial and protozoan pathogens have been successfully cloned, expressed and used as antigens for vaccine development. For example, the major surface antigen of hepatitis B virus, HbsAg, the P subunit of choleratoxin, the enterotoxin of E. coli, the circumsporozoite protein of the malaria parasite, and a glycoprotein membrane antigen from Epstein-Barr virus, as well as tumor cell antigens, have been expressed in various well known vector/host systems, purified and used in vaccines.

A pathologically aberrant cell may also be used in a vaccine composition according to the invention can be obtained from any source such as one or more individuals having a pathological condition or ex vivo or in vitro cultured cells obtained from one or more such individuals, including a specific individual to be treated with the resulting vaccine.

In a particular embodiment, the antigen of the vaccine composition could be a “Tumor associated antigen”. As used herein, the term “tumor associated antigen” refers to an antigen that is characteristic of a tumor tissue. An example of a tumor associated antigen expressed by a tumor tissue may be the antigen prostatic acid phosphatise (see WO 2004026238) or MART peptide T (melanoma antigen).

The vaccine composition according to the invention may contain at least one other immunoadjuvant. A variety of immunoadjuvant may be suitable to alter an immune response in an individual. The type of alteration desired will determine the type of selected immunoadjuvant to be combined with the immunoadjuvant composition of the invention. For example, to enhance the innate immune response, the vaccine composition of the invention can comprise another immunoadjuvant that promotes an innate immune response, such as other PAMP or conserved region known or suspected of inducing an innate immune response. A variety of PAMPs are known to stimulate the activities of different members of the toll-like family of receptors. Such PAMPs can be combined to stimulate a particular combination of toll-like receptors that induce a beneficial cytokine profile. For example, PAMPs can be combined to stimulate a cytokine profile that induces a Th1 or Th2 immune response. Other types of immunoadjuvant that promote humoral or cell-mediated immune responses can be combined with the immunoadjuvant composition of the invention. For example, cytokines can be administered to alter the balance of Th1 and Th2 immune responses. Those skilled in the art will know how to determine the appropriate cytokines useful for obtaining a beneficial alteration in immune response for a particular pathological condition.

In another particular embodiment, the vaccine composition according to the invention, further comprises one or more components selected from the group consisting of surfactants, absorption promoters, water absorbing polymers, substances which inhibit enzymatic degradation, alcohols, organic solvents, oils, pH controlling agents, preservatives, osmotic pressure controlling agents, propellants, water and mixture thereof.

The vaccine composition according to the invention can further comprise a pharmaceutically acceptable carrier. The amount of the carrier will depend upon the amounts selected for the other ingredients, the desired concentration of the antigen, the selection of the administration route, oral or parenteral, etc. The carrier can be added to the vaccine at any convenient time. In the case of a lyophilised vaccine, the carrier can, for example, be added immediately prior to administration. Alternatively, the final product can be manufactured with the carrier.

Examples of appropriate carriers include, but are not limited to, sterile water, saline, buffers, phosphate-buffered saline, buffered sodium chloride, vegetable oils, Minimum Essential Medium (MEM), MEM with HEPES buffer, etc.

Optionally, the vaccine composition of the invention may contain conventional, secondary adjuvants in varying amounts depending on the adjuvant and the desired result. The customary amount ranges from about 0.02% to about 20% by weight, depending upon the other ingredients and desired effect. For the purpose of this invention, these adjuvants are identified herein as “secondary” merely to contrast with the above-described immunoadjuvant composition that is an essential ingredient in the vaccine composition for its effect in combination with an antigenic substance to significantly increase the humoral immune response to the antigenic substance. The secondary adjuvants are primarily included in the vaccine formulation as processing aids although certain adjuvants do possess immunologically enhancing properties to some extent and have a dual purpose.

Examples of suitable secondary adjuvants include, but are not limited to, stabilizers; emulsifiers; aluminum hydroxide; aluminum phosphate; pH adjusters such as sodium hydroxide, hydrochloric acid, etc.; surfactants such as Tween® 80 (polysorbate 80, commercially available from Sigma Chemical Co., St. Louis, Mo.); liposomes; iscom adjuvant; synthetic glycopeptides such as muramyl dipeptides; extenders such as dextran or dextran combinations, for example, with aluminum phosphate; carboxypolymethylene; bacterial cell walls such as mycobacterial cell wall extract; their derivatives such as Corynebacterium parvum; Propionibacterium acne; Mycobacterium bovis, for example, Bovine Calmette Guerin (BCG); vaccinia or animal poxvirus proteins; subviral particle adjuvants such as orbivirus; cholera toxin; N,N-dioctadecyl-N′,N′-bis(2-hydroxyethyl)-propanediamine (pyridine); monophosphoryl lipid A; dimethyldioctadecylammonium bromide (DDA, commercially available from Kodak, Rochester, N.Y.); synthetics and mixtures thereof. Desirably, aluminum hydroxide is admixed with other secondary adjuvants or an immunoadjuvant such as Quil A.

Examples of suitable stabilizers include, but are not limited to, sucrose, gelatin, peptone, digested protein extracts such as NZ-Amine or NZ-Amine AS. Examples of emulsifiers include, but are not limited to, mineral oil, vegetable oil, peanut oil and other standard, metabolizable, nontoxic oils useful for injectables or intranasal vaccines compositions.

Conventional preservatives can be added to the vaccine composition in effective amounts ranging from about 0.0001% to about 0.1% by weight. Depending on the preservative employed in the formulation, amounts below or above this range may be useful. Typical preservatives include, for example, potassium sorbate, sodium metabisulfite, phenol, methyl paraben, propyl paraben, thimerosal, etc.

The vaccine composition of the invention can be formulated as a solution or suspension together with a pharmaceutically acceptable medium.

Such a pharmaceutically acceptable medium can be, for example, water, phosphate buffered saline, normal saline or other physiologically buffered saline, or other solvent or vehicle such as glycol, glycerol, and oil such as olive oil or an injectable organic ester. A pharmaceutically acceptable medium can also contain liposomes or micelles, and can contain immunostimulating complexes prepared by mixing polypeptide or peptide antigens with detergent and a glycoside, such as Quil A.

Liquid dosage forms for oral administration of the vaccine composition of the invention include pharmaceutically-acceptable emulsions, microemulsions, solutions, suspensions, syrups and elixirs. In addition to the active ingredient(s), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the vaccine compositions of the invention for rectal or vaginal administration may be presented as a suppository, which may be prepared by mixing the active ingredient(s) with one or more suitable non-irritating excipients or carriers comprising, for example, cocoa butter, polyethylene glycol, a suppository wax or salicylate and which is solid at room temperature, but liquid at body temperature and, therefore, will melt in the rectum or vaginal cavity and release the active ingredient(s). Formulations of the present invention which are suitable for vaginal administration also include pessaries, tampons, creams, gels, pastes, foams or spray formulations containing such carriers as are known in the art to be appropriate

Vaccine compositions of this invention suitable for parenteral administration comprise the active ingredient(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and non-aqueous carriers that may be employed in the vaccine compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents that delay absorption such as aluminum monostearate and gelatin.

Injectable depot forms are made by forming microencapsule matrices of the active ingredient(s) in biodegradable polymers such as polylactide-polyglycolide. Depending on the ratio of the active ingredient(s) to polymer, and the nature of the particular polymer employed, the rate of release of the active ingredient(s) can be controlled. Examples of other biodegradable polymers include poly(orthoesters) and poly(anhydrides). Depot injectable formulations are also prepared by entrapping the active ingredient(s) in liposomes or microemulsions that are compatible with body tissue. The injectable materials can be sterilized for example, by filtration through a bacterial-retaining filter.

The formulations may be presented in unit-dose or multi-dose sealed containers, for example, ampoules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The amount of antigen and immunoadjuvant composition in the vaccine composition according to the invention are determined by techniques well known to those skilled in the pharmaceutical art, taking into consideration such factors as the particular antigen, the age, sex, weight, species, and condition of the particular animal or patient, and the route of administration.

While the dosage of the vaccine composition depends notably upon the antigen, species of the host vaccinated or to be vaccinated, etc., the dosage of a pharmacologically effective amount of the vaccine composition will usually range from about 0.01 μg to about 500 μg (and in particular 50 μg to about 500 μg) of the immunoadjuvant compound of the invention per dose.

Although the amount of the particular antigenic substance in the combination will influence the amount of the immunoadjuvant compound according to the invention, necessary to improve the immune response, it is contemplated that the practitioner can easily adjust the effective dosage amount of the immunoadjuvant compound through routine tests to meet the particular circumstances.

The vaccine composition according to the invention can be tested in a variety of preclinical toxicological and safety studies well known in the art.

For example, such a vaccine composition can be evaluated in an animal model in which the antigen has been found to be immunogenic and that can be reproducibly immunized by the same route proposed for human clinical testing.

For example, the vaccine composition according to the invention can be tested, for example, by an approach set forth by the Center for Biologics Evaluation and Research/Food and Drug Administration and National Institute of Allergy and Infectious Diseases.

Those skilled in the art will know how to determine for a particular vaccine composition, the appropriate antigen payload, route of immunization, volume of dose, purity of antigen, and vaccination regimen useful to treat a particular pathological condition in a particular animal species.

In a vaccination protocol, the vaccine may be advantageously administered as a unique dose or preferably, several times e.g., twice, three or four times at week or month intervals, according to a prime/boost mode. The appropriate dosage depends upon various parameters.

As a general rule, the vaccine composition of the present invention is conveniently administered orally, parenterally (subcutaneously, intramuscularly, intravenously, intradermally or intraperitoneally), intrabuccally, intranasally, or transdermally, intralymphatically, intratumorally, intravesically, intraperitoneally and intracerebrally. The route of administration contemplated by the present invention will depend upon the antigen.

The present invention relates to a kit comprising an immunoadjuvant composition as defined above and at least one antigen.

More particularly, the invention relates to a kit comprising:
an immunoadjuvant composition as defined above,
at least one antigen as defined above;

as combined preparation for simultaneous, separate or sequential use to induce a protective immune response against, for example, a pathogen, or to efficaciously protect the subject or the animal against infection.

The immunoadjuvant composition can be administered prior to, concomitantly with, or subsequent to the administration of at least one antigen to a subject to induce a protective immune response against, for example, a pathogen, or to efficaciously protect the subject or the animal against infection. The immunoadjuvant composition and the antigen are administered to a subject in a sequence and within a time interval such that the immunoadjuvant composition can act together with the antigen to provide an increased immune response against said antigen than if they were administered otherwise. Preferably, the immunoadjuvant composition and antigen are administered simultaneously to the subject. Also preferably, the molecules are administered simultaneously and every day to said patient.

A further aspect of the invention relates to a method for vaccinating a subject in need thereof comprising administering a pharmacologically effective amount of an antigen and a pharmacologically effective amount of an immunoadjuvant composition according to the invention.

As used herein, the term “subject” denotes a mammal, such as a rodent, a feline, a canine, and a primate. Preferably a subject according to the invention is a human.

A pharmacologically effective amount of the immunoadjuvant composition according to the invention may be given, for example orally, parenterally or otherwise, concurrently with, sequentially to or shortly after the administration of the antigen in order to potentiate, accelerate or extend the immunogenicity of the antigen.

The dosage of the vaccine composition will be dependent notably upon the selected antigen, the route of administration, species and other standard factors. It is contemplated that a person of ordinary skill in the art can easily and readily titrate the appropriate dosage for an immunogenic response for each antigen to achieve the effective immunizing amount and method of administration.

A further object of the invention relates to a method for inducing the development of T Follicular helper cells (Tfh) in a subject in need thereof comprising administering a pharmacologically effective amount of an immunoadjuvant composition according to the invention.

A further object of the invention relates to a MyD88-dependent pathway agonist for enhancing the clinical efficacy of an adjuvant in a subject in need thereof. As used herein the expression “a method for enhancing the clinical efficacy of an adjuvant” refers to the fact that the MyD88-dependent pathway agonist of the invention improves the settlement of the humoral response (production of antibody against a specific antigen) boosted by the adjuvant. According to the invention, the MyD88-dependent pathway agonist potentiates the activity of the adjuvant for development of the humoral response. The term “potentiate”, as used herein, means to enhance or increase at least one biological effect or activity of the adjuvant so that either (i) a given concentration or amount of the adjuvant results in a greater biological effect or activity when the adjuvant is potentiated than the biological effect or activity that would result from the same concentration or amount of the adjuvant when not potentiated; or (ii) a lower concentration or amount of the adjuvant is required to achieve a particular biological effect or activity when the adjuvant is potentiated than when the adjuvant is not potentiated; or (iii) both (i) and (ii).

In some embodiments, the MyD88-dependent pathway agonist and the adjuvant are to be used simultaneous or sequentially within a given time. The adjuvant can be applied in either order, e.g. the adjuvant can be applied first and then the MyD88-dependent pathway agonist can be applied or vice versa. It is obvious that when a composition comprising both the adjuvant and MyD88-dependent pathway agonist (as above described) is used both components will be applied at the same time. When used sequentially, different routes of administration could be envisaged.

In another embodiment, the immunoadjuvant composition according to the invention may be used to treat or prevent infectious disease or cancer disease.

In one embodiment, the infectious disease can be Influenza or toxoplasma gondii infection. In this case, the immunoadjuvant composition can contain some antigen specific of the pathogen or the attenuated pathogen itself.

In another embodiment, the immunoadjuvant composition according to the invention may be used to treat or prevent animal disease. In this way, the immunoadjuvant composition may be used in the veterinary field.

The invention will be further illustrated by the following figures and examples. However, these examples and figures should not be interpreted in any way as limiting the scope of the present invention.

### FIGURES

FIG. 1: MyD88-dependent TLR agonists (LPS and CpG) promote Ag-specific Tfh cell development.

(A) 9 days after s.c. immunisation with 40 μg of 1W1K in IFA only or IFA with poly(I:C), LPS or CpG)(n≧5/group, mean±SEM), dLN (inguinal and periaortic) were collected and analysed for the detection of 1W1K-specific activated CD4+ T cells (Tetramer+CD44+). Frequency of Tfh cells among 1W1K-specific CD4+ T cells in dLN 9 days after immunisation with different dose of CpG (B) or with adjuvant (Alum, SAS) mixed with CpG (C). ns, non significant; **p≦0.01; ***p≦0.005; ****p≦0.001

FIG. 2: Addition of CpG to adjuvant enhances OVA-specific B cell responses.

14 days after s.c. immunisation of C57Bl/6 mice with 100 μg of OVA in IFA, dLN and sera were collected and analysed to estimate OVA-specific B cells and Ig levels, respectively. (A): Numbers of total OVA-specific B cells, OVA-specific GC-B cells or OVA-specific PC in dLN after immunisation with IFA with CpG relative to numbers found in OVA in IFA only immunised animals (n≧5/group; mean±SEM). (B): Levels of OVA-specific IgG in sera of mice 14 days after immunisation with OVA in IFA or IFA with poly(I:C), LPS or CpG (n≧5 mean±SEM). OVA-specific IgG are estimated as the difference between the Optical Density (OD) obtained by ELISA with sera collected 14 days after immunisation subtracted to value obtained for sera collected just before immunisation, calculated individually for each mouse. (C): Same as (B) using either Alum; Alum with CpG or SAS; SAS with CpG (n=4/group, mean±SEM). ns, non significant; *p≦0.05; **p≦0.01

FIG. 3: Impact of CpG on Tfh cell differentiation relies on CD11c+ cells.

(A) and (B): 8 weeks after reconstitution, chimeric CD11 cDTR+wt or TLR9KO or MyD88KO or TRIF KO C57Bl/6 mice were treated every 2 days with DTx and immunised with 1W1K or OVA in IFA or IFA with CpG. Frequency of Tfh among 1W1K-specific CD4+ T cells cells in dLN 9 days after immunisation (n=5/group, mean±SEM) and levels of OVA-specific IgG, in the sera of mice 14 days after immunisation (n=5/group, mean±SEM)(F). ns, non significant; *p≦0.05; **p≦0.01; ****p≦0.001

FIG. 4: Antigen-presenting CD11b+moDC produce IL-6 in response to CpG.

Total cell count number of CD11c+ beads+IL-6+ cells in dLN of mice 2 days after immunisation with 10¹⁰ Ea-coated beads in IFA or IFA complemented 1, 2, 10, 50 or 100 μg of CpG (n≧3/group, mean±SEM). ns, non significant; *p≦0.05; **p≦0.01

FIG. 5: IL-6 produced by Ag-presenting DC in response to CpG promotes Tfh cell differentiation.

(A): Frequency of Tfh among 1W1K-specific CD4+ T cells was estimated and presented with open bars for IFA immunised animals and solid bars for IFA with CpG. (B): 8 weeks after reconstitution, chimeric CD11cDTR+wt→C57Bl/6 (wt) and CD+IL-6KO→C57Bl/6 (IL-6KO) mice were immunised with 1W1K in IFA or IFA with CpG. Frequency of Tfh among 1W1K-specific CD4+ T cells in dLN 9 days after immunisation from IFA immunised chimeric mice treated three times with DTx (n≧4/group, mean±SEM). ns, non significant; **p≦0.01

FIG. 6: monocyte-derived DC drive the increase of Tfh cell development due to CpG.

(A): WT animals were immunized with 1W1K or OVA in IFA or IFA with CpG and treated twice with liposomes containing either PBS or clodronate. 9 days after immunisation with 1W1K and the frequency of Tfh among 1W1K-specific CD4+ T cells was estimated in the corresponding animals (n≧4/group, mean±SEM). (B) 14 days after immunisation with OVA, total OVA-specific IgG were estimated by ELISA (n≧5/group, mean±SEM)(B). CX3CR1KO and wt littermates or chimeric wt C57Bl/6 (wt) and CCR2KO→C57Bl/6 (CCR2KO) mice 8 weeks after reconstitution were immunised with 1W1K in IFA or IFA with CpG. 9 days after immunisation, the frequency of Tfh among 1W1K-specific CD4+ T cells was estimated in the corresponding animals (n=5/group, mean±SEM). ns, non significant; *p≦0.05; **p≦0.01

FIG. 7: CpG promotes Ag-specific Tfh cell development in a dose dependent-manner.

9 days after s.c. immunisation with 40 μg of 1W1K in RIBI with or without CpG, dLN (inguinal and periaortic) were collected and analysed for the detection of Th cells (CD4+)(A), 1W1K-specific activated CD4+ T cells (Tetramer+CD44+)(B). Frequency of Tfh cells among 1W1K-specific CD4+ T cells in dLN 9 days after immunisation with different dose of CpG (C).

FIG. 8: Addition of CpG promotes Ag-specific Tfh cell development and allows to decrease Ag dose

9 days after s.c. immunisation with different dose of 1W1K in RIBI with 50 μg/mL of CpG, dLN (inguinal and periaortic) were collected and analysed for the detection of Th cells (CD4+)(A), 1W1K-specific activated CD4+ T cells (Tetramer+CD44+)(B). Frequency of Tfh cells among 1W1K-specific CD4+ T cells in dLN 9 days after immunisation with different dose of 1W1K peptide (C).

FIG. 9: More Antigen-presenting moDC in response to CpG

Frequency of YAe+cDC (CD11c+CD8a− CD11b+CD64− YAe+)(A) and of YAe+moDC (CD11c+CD8a− CD11b+CD64+YAe+) in dLN of mice 2 days after immunisation with 100 μg of Ea in RIBI complemented with 0, 5, 10, 50 μg of CpG.

### EXAMPLE

### Material & Methods

Mice

C57Bl/6 were from Janvier, JHT were kindly provided by Dr S. Fillatreau, TLR9KO and TRIFKO by Dr E. Barhaoui, IL-6K0 by Dr H. Coppin, CD11c-DTR and CD45.1 C57Bl/6 by Dr S. Guerder, MyD88KO and CX3CR1KO by Dr R. Burcelin and CCR2KO by Dr T. Walzer. All mice were maintained under pathogen-free conditions at CHU Purpan. All experimental mice were females used at 8-16 weeks of age and were age-matched (within 2 weeks) within experiments. The Institutional Animal Care and Use Committee reviewed and approved all experiments.

Reagents

Ovalbumin protein (OVA) was from Sigma-Aldrich, Ea52-68, Ea52-68-FITC, 1W1K were from Genecust. SAS and IFA were from Sigma-Aldrich, Alum and OVA-Alexa488 from Invitrogen, CpG, LPS and poly(I:C) were from Invivogen. For construction of microsphere-linked Ag, 0.5 μm of carboxylated green fluorescent latex microspheres were purchased from Polysciences (Warrington, Pa.). Protein or peptide were covalently linked to microspheres using carbodiimide (EDAC)-based chemistry (as directed by Polysciences).

Immunisation

Mice were either i.p. injected or immunized s.c. at the base of tail with 40 μg of peptide 1W1K, 100 μg of OVA, 200 μg of Ea-FITC in the indicated adjuvant with or without soluble TLR agonist, in a final volume of 2004. To target moDC, mice were immunised s.c. with 1.2×1010 beads (which correspond to 40 μg Ea52-68, 1W1K or 100 μg OVA) in indicated adjuvant with or without soluble TLR agonist. At indicated times post-immunisation, spleen or dLN (inguinal and periaortic) were collected and cells were stained for flow cytometry analysis.

In Vivo Treatment

To block IL-6 signalling, i.p. injections of 100 μg D7715A7 mAb (anti-IL-6R□, eBioscience) or Rat IgG2b isotype control were performed at day −1 and +4 post-immunisation. To deplete mice of monocytes, i.v. injections of 250 μL of clodronate liposomes or control PBS liposomes (clodronateliposome.com) were performed from day −1 every second day.

ELISA

To determine the amount of OVA-specific Ig isotypes in serum, 96-well ELISA plates (Thermo scientific) were coated overnight with 10 μg/ml OVA in PBS, followed by protein saturation with PBS/Tween20 0.05%/skimmed milk 3% before incubation with pre-diluted sera. Total Ag-specific IgG were detected with horseradish peroxidase (HRP)-conjugated anti-mouse total IgG (Southern Biotech). The HRP substrate o-Phenylenediamine dihydrochloride (OPD) was purchased from Sigma-Aldrich. Ab presence was determined as the subtraction of optical density value at 490 nm of sera before and 14 days post-immunisation. IL-6 in sera was determined using IL-6 ELISA kit (eBioscience).

Flow Cytometry

For population analysis, dLN were harvested from mice and single cell suspensions were prepared in PBS with 2% FCS, 5 mM EDTA. For DC analysis, lymphoid organs were dissociated using 125 μg/mL LiberaseTL (Roche) and 40 μg/mL DNAase I (Sigma-Aldrich) at 37° C. for 20 min. 1×108 cells/ml were labelled at 4° C. for 45 min with mAbs: Reagents from BD Pharmingen [AlexaFluor488-K112-91 (anti-Bcl-6); PE-M1/70 (anti-CD11b), PE-281-2 (anti-CD138); Cy5PE-M1/70 (anti-CD11b); APC-2G8 (anti-CXCR5); V500-500A2 (anti-CD3); Pacific Blue-53-6.7 (anti-CD8α); Biotin-Jo2 (anti-CD95); Biotin-2G8 (anti-CXCR5)], Reagents from eBioscience [FITC-eBio1D3 (anti-CD19), FITC-RA3-6B2 (anti-B220), FITC-RM4-5 (anti-CD4); PE-MAR-1 (anti-FceR1), PE-205yekta (anti-CD205), PE-2E7 (anti-CD 103), PE-MP5-20F3 (anti-IL-6); Cy5PE-RM4-5 (anti-CD4); Cy7PE-N418 (anti-CD11c), Cy7PE-53-6.7 (anti-CD8α); Cy5.5PE-anti-CD19, Cy5.5PE-HK1.4 (anti-Ly-6c); APC-N418; AlexaFluor647 anti-GL-7; AlexaFluor700-MEL-14 (anti-CD62L), AlexaFluor700-eBio1D3, AlexaFluor700-IM7 (anti-CD44); APCeFluor780-IM7, APCeFluor780-BM8 (anti-F4/80), APCeFluor780-RA3-6B2; eFluor450-11-26c (anti-IgD), eFluor450-MEL-14; biotin-Y-Ae], reagent from R&D PE-475301(anti-CCR2). Before data collection 2 gg/m1 propidium iodide (PI) was added to samples. 1W1K-IAb tetramer was obtained from NIH Tetramer core facility. For IL-6 intracellular staining cell suspensions were incubated for 4 hr at 37° C. in the presence of 3 μg/mL Golgi Plug (BD Bioscience) and 4 μM Monensin, fixed and permabilised using BD Fixation/Permeabilisation kit. Before permeabilisation cells were stained with Fixable Viability Dye eFluor450 or eFluor660 (eBioscience). Data were collected on a BD LSRII™ (BD Biosciences) and analysed using FlowJo software (Tree Star).

BM Chimeras

6-8-wk-old C57Bl/6 mice were irradiated (0.85 Gy) the day before i.v. injection of 1×107 T cell-depleted BM cells that consisted of a mix of 70% from CD11c-DTR or JHT supplemented with 30% from TLR9KO, Myd88KO, TRIFKO, IL-6K0 or wt CD45.1 C57Bl/6 mice. After 8 wk, the chimeras with CD11c-DTR BM were i.p. treated every second day with 4 ng/g body weight DTx (Sigma-Aldrich) and 1 day before immunisation with 20 ng/mouse s.c. Treated animals were then s.c. immunised either with 1W1K or OVA as described above.

Statistical Analysis

Mean values, standard error means (SEM), Student's t-test (paired and unpaired) or the Mann-Whitney U-test were calculated with GraphPad Prism (GraphPad Software). All p values of 0.05 or less were considered significant.

Results

MyD88-Dependent TLR Agonists (LPS and CpG) Promote Ag-Specific Tfh Cell Development

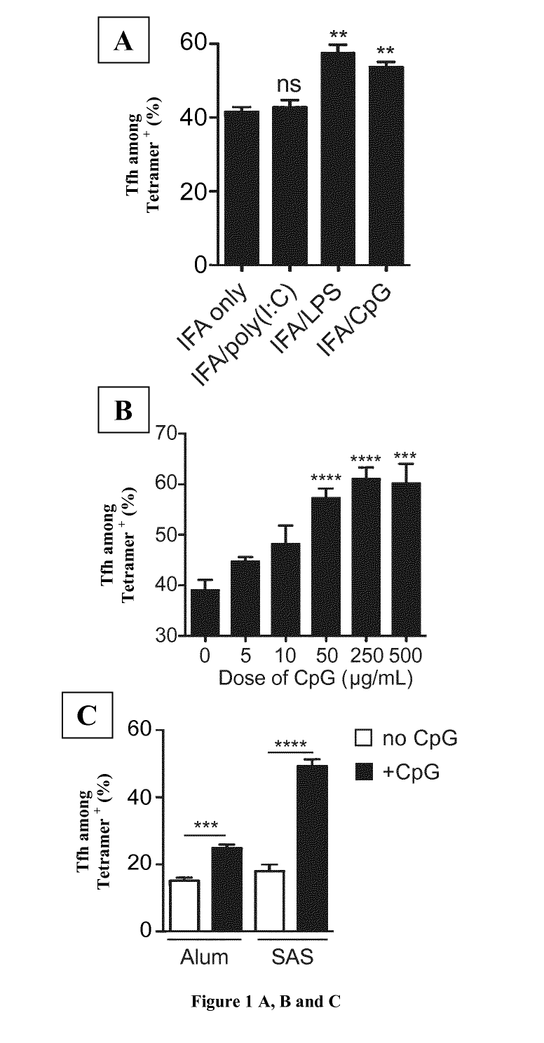

The combination of adjuvant to a peptide favours robust T-dependent B cell immunity. To test whether addition of TLR agonists to other vaccine adjuvant could have a synergistic effect on the induced immune response, we examined the I-Ab restricted murine CD4+ T cell response to a peptide variant (EAWGALANKAVDKA, called 1W1K peptide hereafter, SEQ ID NO:1) of the I-E alpha chain immunodominant peptide 52-68 (Ea52-68) in C57Bl/6 mice. We followed the 1W1K-specific CD4+ T cells with the corresponding pMHCII tetramer in the draining lymph nodes (dLN) after sub-cutaneous (s.c.) immunisation in Incomplete Freund's adjuvant (IFA). At the peak of the effector CD4+ T cell response, we detected the CD44+ and pMHCII tetramer+1W1K-specific CD4+ T cells in the dLN (data not shown). We can further analyse the 1W1K-specific CD4+ T cells that were CD62L10 CXCR5+ and that correspond to Tfh cells, as confirmed by their expression of Bcl-6 (data not shown). We found that addition of poly(I:C), CpG, or LPS to IFA did not statistically change the total number of CD4+ T cells in the dLN (PI-CD19-CD8-CD4+, data not shown) and the amount of 1W1K-specific CD4+ T cells (data not shown) at the peak of the primary response. Strikingly, we observed that addition of LPS and CpG, the MyD88-dependent TLR agonists, to IFA induced an increase of 1W1K-specific Tfh cells in the dLN (FIG. 1A). In contrast, addition of poly(I:C), the TRIF-dependent TLR agonist, had no enhancing effect on the Tfh compartment (FIG. 1A). Moreover, no variation was found in the kinetics of the 1W1K-specific CD4+ T cell response in both immunising conditions (data not shown) and the enhancing effect due to CpG addition to IFA on the Tfh compartment was observed at all time points tested from day 5 after immunisation (data not shown). Interestingly, we observed that the increased differentiation of 1W1K-specific Tfh cells mediated by CpG was dose-dependent and reached a plateau for a dose of 250 μg/mL (FIG. 1B). As the adjuvanticity of CpG may vary with the physical context in which it is presented to TLR9, we next tested whether CpG also enhances Tfh cell responses when added to two other types of adjuvant, Aluminium-containing adjuvant (Alum) and Sigma Adjuvant System (SAS). While IFA is a ‘water-in-oil’ adjuvant, Alum is an aqueous adjuvant and SAS is an ‘oil-in-water’ adjuvant composed of MPL and Trehalose Dicorynomycolate in metabolisable squalene oil. We found that CpG addition to Alum and SAS also increased 1W1K-specific Tfh cells (FIG. 1C). Altogether, these results demonstrate that addition of soluble MyD88-dependent but not of TRIF-dependent TLR agonist to other vaccine adjuvant enhances the pool of Ag-specific Tfh cells in vivo without affecting the overall magnitude and the dynamics of the Ag-specific CD4+ T cell compartment.

MyD88-Dependent TLR Agonists (LPS and CpG) Boost Ag-Specific B Cell Responses

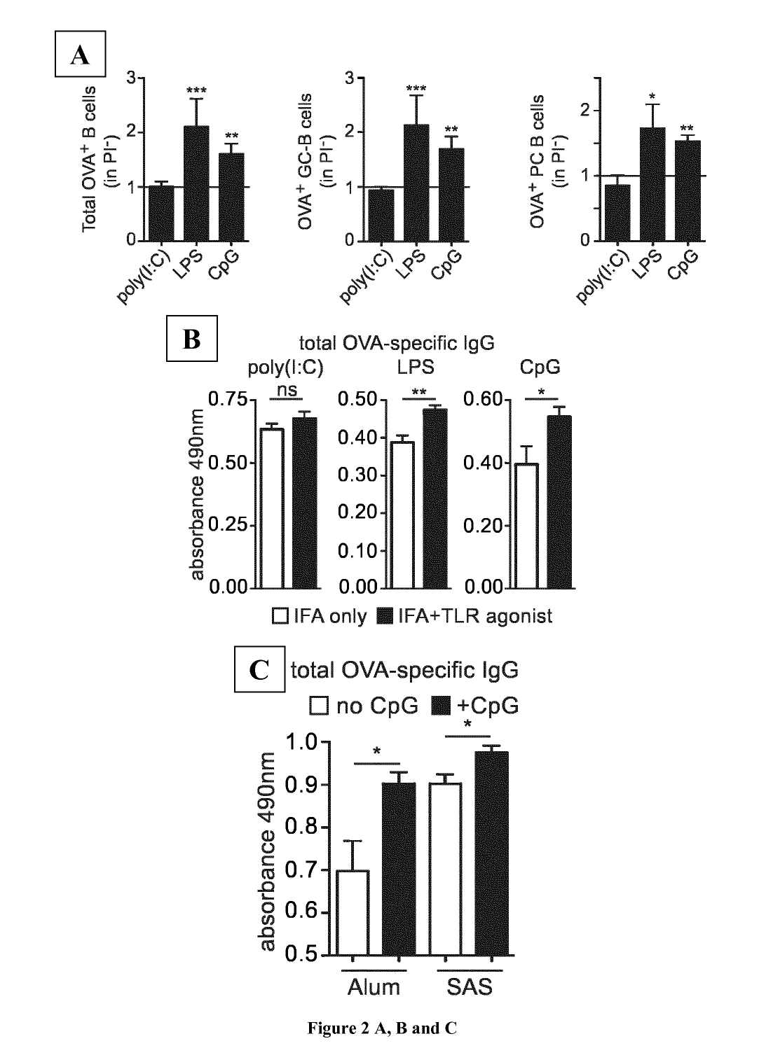

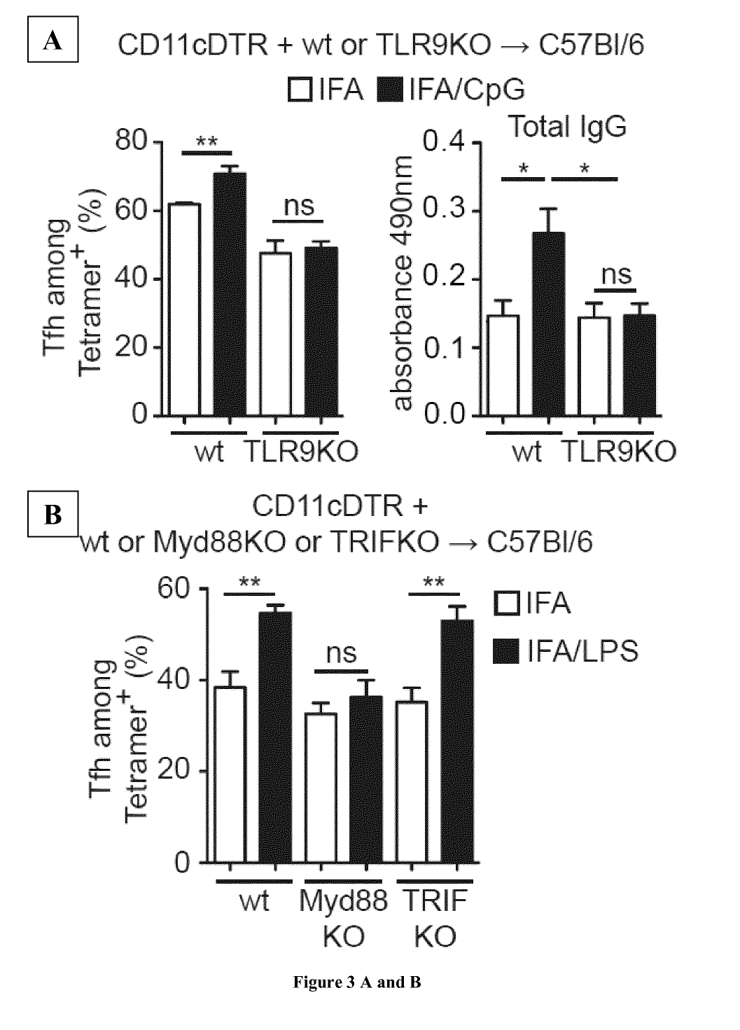

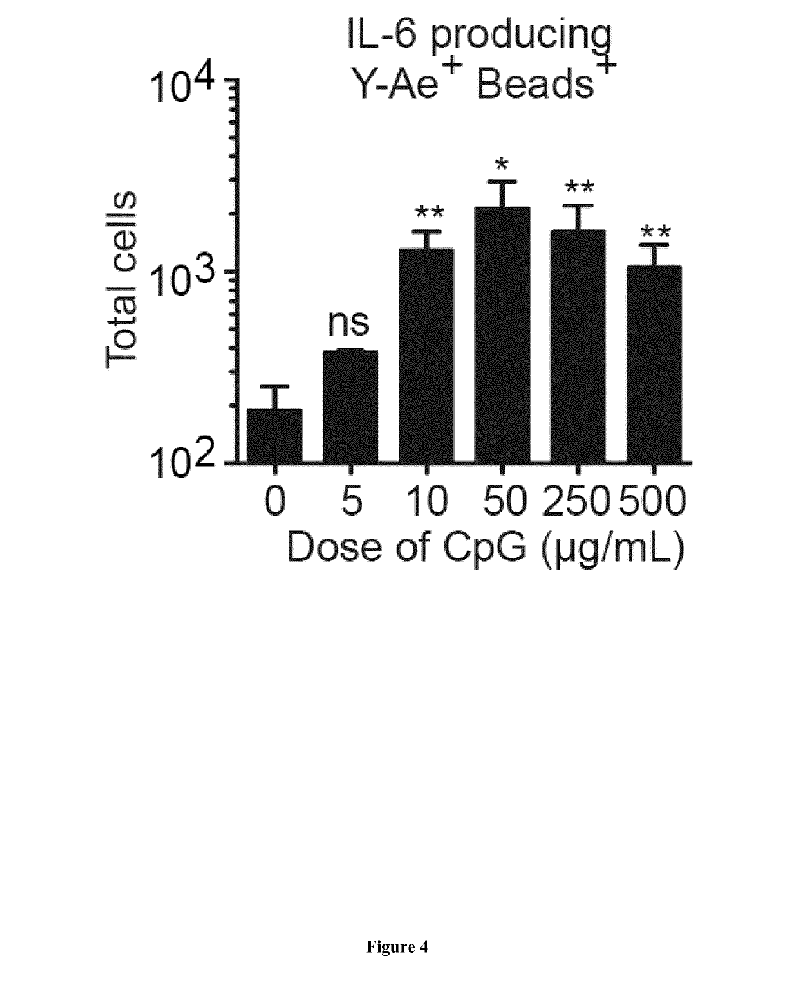

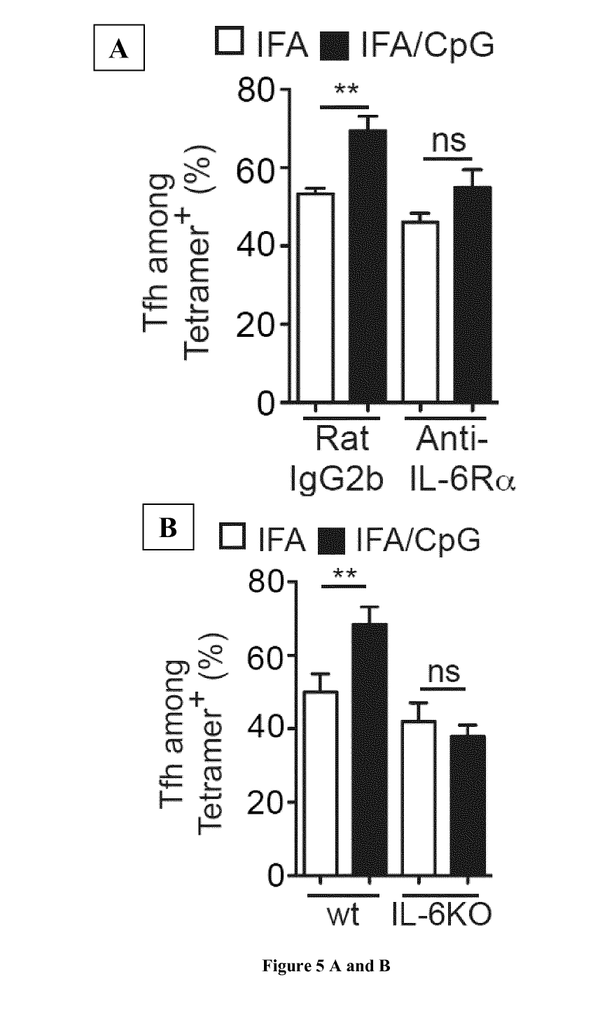

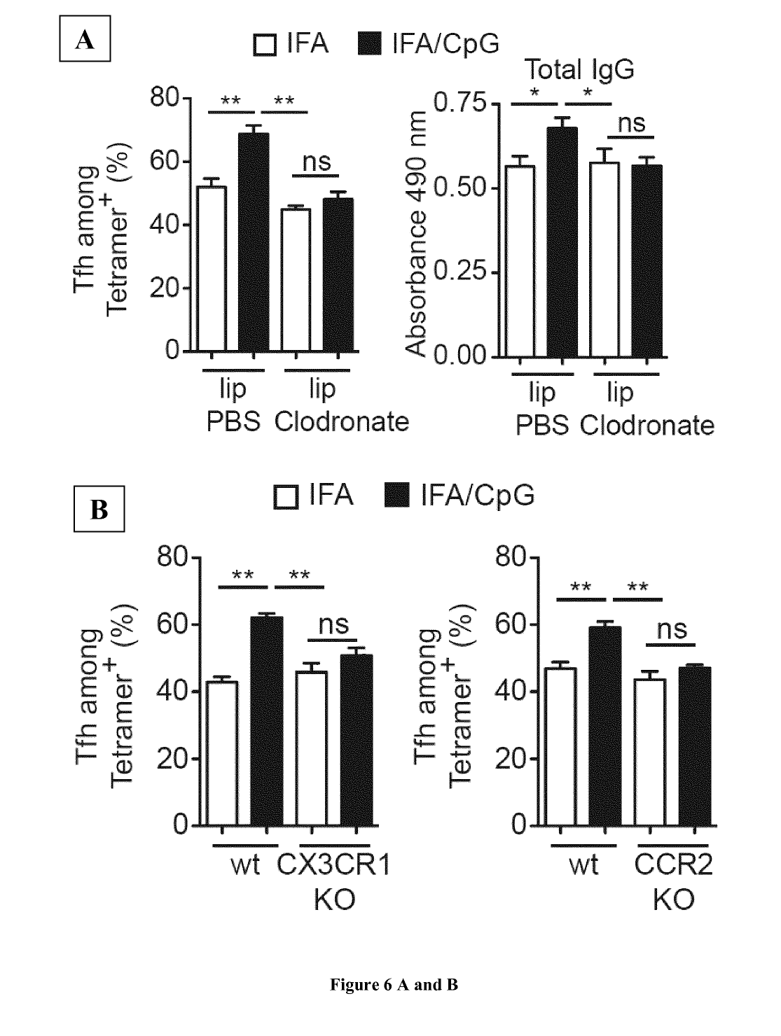

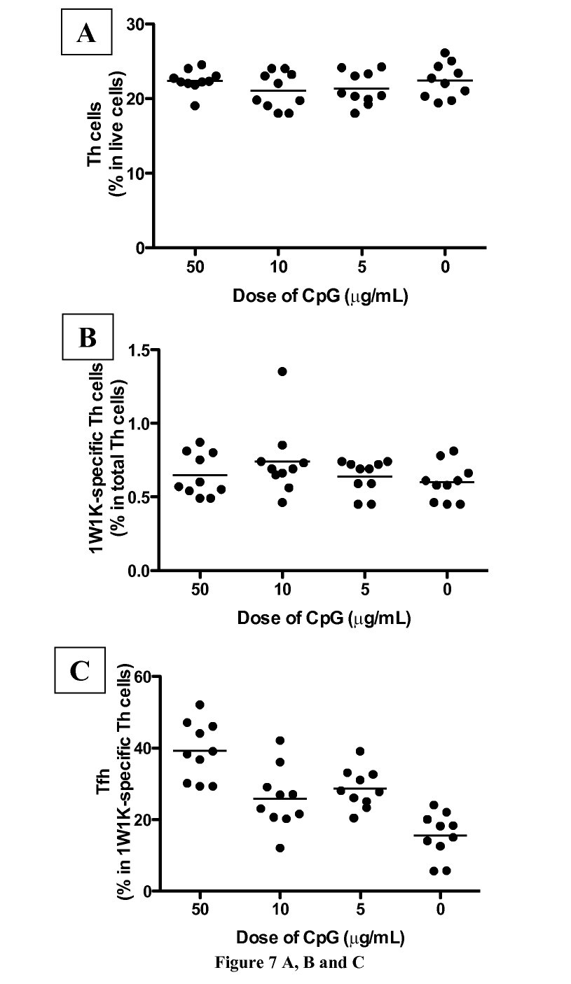

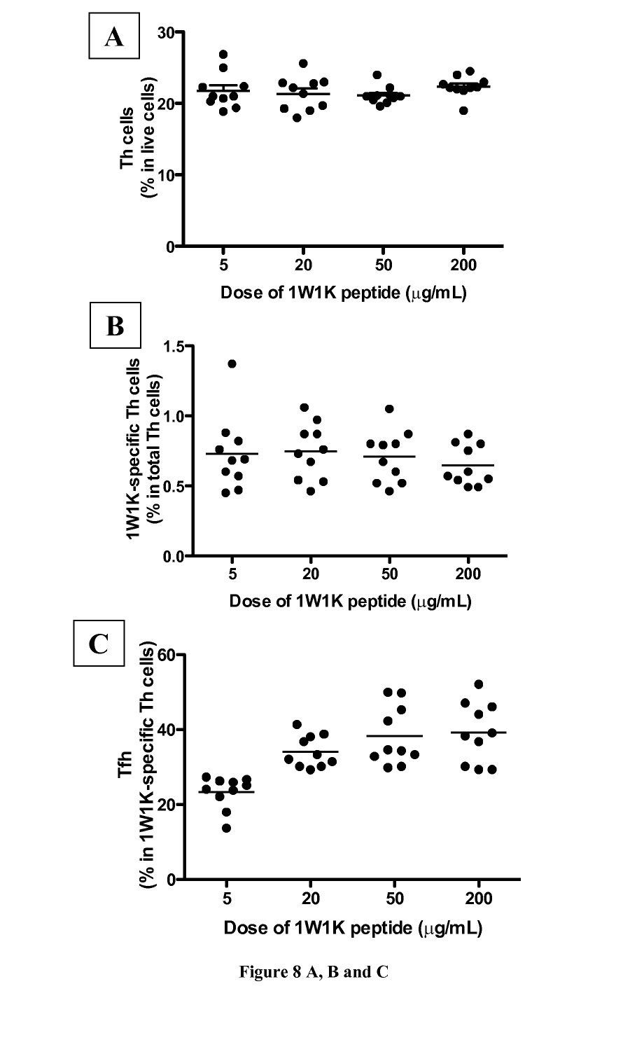

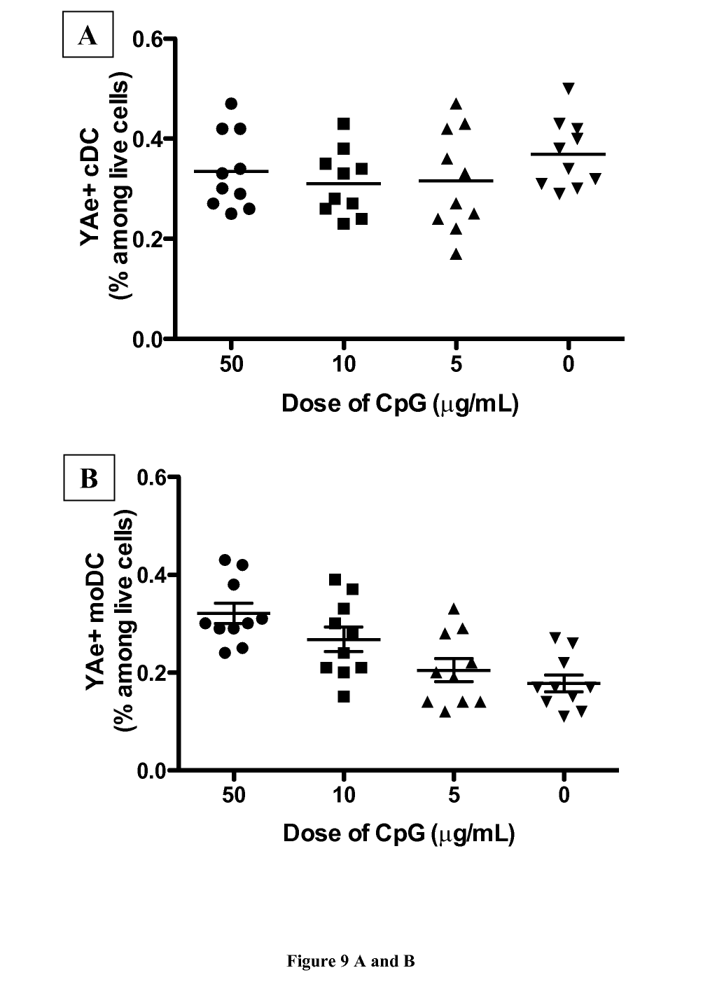

We hypothesized that an increase in the CD4+ T cell help and specifically an increase of the Tfh cells, the critical regulators of B cell responses, could result in a greater B cell response to the Ag after addition of TLR agonist to another adjuvant. The murine B cell response to ovalbumin (OVA) in wt animals is a valuable immunisation model that can be monitored via the polyclonal Ig repertoires. OVA-specific B cells can be detected by flow cytometry as CD3-IgD− cells that bind specifically to A1exa488-conjugated OVA (OVA+) (data not shown). Two functionally distinct populations can be examined upon phenotypic analysis: CD138+PC and CD138-B220+GL-7+CD95+GC-B cells (data not shown). Using this strategy, we determined if addition of TLR agonist to IFA impacts OVA-specific B cell responses in vivo. As observed for the Ag-specific Tfh cells, we observed that addition of LPS or CpG but not of poly(I:C) to IFA increased the overall OVA-specific B cells and among them GC-B cells and PC (FIG. 2A). Moreover, we found a significant increase in circulating OVA-specific IgG in animals immunised with IFA supplemented with CpG or LPS but not with poly(I:C) (FIG. 2B). Furthermore, circulating OVA-specific IgG were also increased by addition of CpG to either Alum or SAS (FIG. 2C). Interestingly, we also found that increase of Tfh-dependent B cell responses after CpG addition could be observed not only at the peak of the immune response but also at later time points after immunisation as shown 60 days after immunisation for the 1W1K-specific Tfh cell responses and 30 and 60 days after immunisation for the OVA-specific IgG response (data not shown). Altogether, these data demonstrate that addition of soluble MyD88-dependent TLR agonists (LPS or CpG) to other vaccine adjuvant can intensify specifically Ag-specific Tfh-dependent Ab responses in vivo.

Impact of TLR agonists on B cell responses is B cell-extrinsic CpG adjuvanticity on T-dependent Ab response to protein was shown to be driven in vivo by CD11c+DC when CpG is soluble and by B cells when it is contained in virus like particle. Moreover, while Tfh cells control B cell maturation, interactions with B cells are reciprocally essential for Tfh differentiation. Finally, LPS and CpG can induce cytokine production by B cells such as IL-6 that is known to possibly promote Tfh cell differentiation. Thus, we next explored whether the increase of Ag-specific Tfh cell and B cells upon CpG or LPS addition to other adjuvant is DC- or B-cell mediated. To test this hypothesis, a bone marrow (BM) chimeric system in which TLR9 deficiency was restricted to B cells was first developed. We took advantage of the JHT mouse model that totally lacks functional B cells as a result of a genetic deletion of the heavy chain joining region. C57Bl/6 recipients were lethally irradiated before reconstitution with 70% JHT BM supplemented with 30% BM from TLR9KO mice. The resulting BM chimeras were immunised with 1 W1K in IFA or IFA with CpG and the 1W1K-specific Tfh cell response was monitored. We found that absence of TLR9 signalling in B cells had no effect on the enhancing effect of Tfh development due to CpG addition (data not shown). Similarly, when chimeric animals were immunised with OVA, we showed that the increase of the OVA-specific IgG due to CpG addition was also observed even in absence of TLR9 signalling in B cells (data not shown). Then, we tested whether it was also true for LPS. In this order, we lethally irradiated C57Bl/6 recipients before reconstitution with 70% JHT BM supplemented with 30% BM from MyD88KO mice. The resulting BM chimeras were immunised either with 1W1K or with OVA and the enhancing effects due to LPS addition to IFA on 1W1K-specific Tfh cells (data not shown) and on OVA-specific IgG (data not shown) were also observed even in absence of MyD88 signalling in B cells. Finally, we intra-peritoneal (i.p.) immunized JHT and wt littermate mice with 1W1K in Alum or Alum with CpG or LPS. 5 Days after immunisation, we found that CpG and LPS addition to Alum enhanced 1W1K-specific Tfh cell differentiation even in the absence of functional B cells (data not shown). Moreover, we measured IL-6 in the serum of immunised animals 4 hours after i.p. injection and observed a significant decrease in CpG- and LPS-sensitised JHT animals when compared to sensitised wt mice showing that B cells, as expected, secrete large amount of IL-6 in response to CpG or LPS (data not shown). Altogether these data show that the increase in Tfh cell development and B cell responses upon addition of CpG or LPS to other vaccine adjuvant does not depend on TLR-signalling in B cells.

CD11c+Cells Mediate the Increase of T-Dependent B Cell Response Due to TLR Agonist

To address whether CD11c+DC cells drive the effect of TLR agonist addition to other adjuvant on T-dependent B cell response, a BM chimeric system in which TLR9, MyD88 or TRIF deficiency was restricted to CD11c+ cells was developed. For this purpose, we used a tg mouse model of CD11c-DTR in which CD11c+ cells can be depleted after diphtheria toxin (DTx) injection. As described above, C57Bl/6 recipients were lethally irradiated before reconstitution with BM from CD11c-DTR and TLR9KO mice. The resulting BM chimeras were treated with DTx, which resulted in the specific depletion of CD11c+ cells from CD11c-DTR origin, and were immunised with 1W1K in IFA or IFA with CpG. We found that absence of TLR9 signalling in CD11c+DC resulted in the absence of Tfh differentiation enhancement due to CpG (FIG. 3A). Similarly, when these chimeric animals were treated with DTx and immunised with OVA in IFA or IFA with CpG, we showed that the increase of OVA-specific IgG due to CpG addition was observed only in presence of TLR9 signalling in CD11c+ cells (FIG. 3A). Finally, other chimeric animals reconstituted with BM from CD11c-DTR supplemented with BM from wt, MyD88KO or TRIFKO animals showed that the increase of 1W1K-specific Tfh response upon addition of LPS was not observed when MyD88 signalling in CD11c+ cells was impaired (FIG. 3B). Thus, the increase of T-dependent B cell response upon addition of soluble CpG or LPS to other adjuvant relies on TLR signalling in CD11c+ cells.

Impact of CpG on Tfh Cell Differentiation is Independent of Plasmacytoid DC

IL-6, TNF-α or type I IFN production by pDC can be induced upon ligation of TLR9. Moreover, after protein immunisation, type I IFN secreted by pDC induces a signalling cascade in cDC that leads to the induction of Tfh cell differentiation. Thus, CpG could enhance Tfh cell differentiation in vivo either directly or indirectly through cytokine production by pDC. On one hand, it has been shown by Sapoznikov and colleagues that DTx treatment in mix BM chimera using CD11c-DTR BM as we described in the experiment above (FIGS. 3A and B) did not deplete plasmacytoid CD11c+DC while other CD11c+ cells were [Sapoznikov et al., 2007]. Therefore, it is likely that pDC play a minor role in the effect we observed after CpG addition to IFA. Still, we treated mice on days −1, +1 and +3 (relative to immunisation with 1W1K) with 200 μg of the 120G8 monoclonal Ab (mAb), which depletes selectively pDC, or with a Rat IgG1 isotype control. At day +5, we sacrificed the mice and ensured that the 120G8 injection had caused a significant decrease of pDC (PI-CD11c+Ly6C+B220+) in the spleen (data not shown). The depletion was, however, not complete, but this was expected since the last treatment with pDC-depleting mAb was performed 2 days before we sacrificed the mice. This decrease in splenic pDC numbers correlated with a significant decrease of IL-6 in the serum of 120G8-treated animals showing that pDC, as expected, secrete large amount of IL-6 in response to CpG (data not shown). Nevertheless, it was striking that, regardless of whether pDC were present (isotype control-treated mice) or depleted (120G8-treated animals), addition of CpG had a marked positive effect on 1W1K-specific Tfh cell differentiation (data not shown). Taken together, these results strongly suggest that pDC do not play a role in the increase of Tfh-dependent B cell responses due to soluble CpG addition to vaccine adjuvant.

CD11b+Conventional DC and Monocyte-Derived DC Present the Ag to CD4+ T Cells in the dLN after Immunisation

Our above results indicate that the promoting effect of CpG is dependent on CD11c+

DC and that this activity is independent of pDC. To track Ag-presenting DC, we took advantage of two technical approaches allowing us to follow and isolate directly the Ag-presenting DC in wt animals after immunisation. First, to be able to track cells that had captured Ag, we conjugated it directly to FITC, a fluorescent molecule. Second, we used the Y-Ae mAb that specifically recognizes the pMHCII complex I-Ab-Ea52-68 in C57Bl/6 mice. 2 days after immunisation with Ea-FITC, we found that a majority of the CD11c+DC in the dLN that had captured the Ag (CD11c+FITC+, FIG. 4A) were Y-Ae+, irrespective of CpG addition to IFA (data not shown). Alternatively, if we immunised animals with OVA-FITC, the CD11c+DC that had captured the Ag were all Y-Ae− (data not shown). We next examined the dynamics of Ag-presenting DC in dLN after immunisation with Ea-FITC in IFA or IFA with CpG and found that they were similar in presence or absence of CpG with a peak 48 hours after immunisation (data not shown). DC express CD11c and MHCII molecules, and have been categorized as CD8α+ and CD11b+-type DC, a dichotomy that takes into account phenotypic and functional attributes. CD11c+FITC+Y-Ae+DC were mainly CD11b+CD8α− (data not shown). Extensive phenotypic analyses of these latter cells showed that Ag-presenting CD11b+DC could be divided in cDC and moDC based on CD64 expression (FIG. 4E) with a majority of moDC at 48 hours after immunisation (data not shown).

CpG Promotes IL-6 Secretion by CD11b+Monocyte-Derived DC

We explored the mechanism by which CpG-sensitised Ag-presenting DC could induce more Tfh cells in vivo. We performed intracellular staining and found that CD11c+FITC+DC secreted more IL-6 in animals immunised with IFA with CpG than those with IFA control animals (data not shown), 24 and 48 hours after immunisation (data not shown). Strikingly, the totality of these cells was CD64+ so they could be categorized as moDC (data not shown). We thus took advantage of the phagocytic capacity of these cells and targeted these cells by using Ea52-68 peptide conjugated to large nanoparticles of 0.5 μm (called beads hereafter). 2 days after immunisation with Ea-beads in IFA, we found that all the CD11c+DC in the dLN that had captured the beads were Y-Ae+(data not shown). Alternatively, if we immunised animals with OVA-conjugated beads, the CD11c+DC that had captured the beads were all Y-Ae− (data not shown). As expected and in contrast to what observed after Ea-FITC immunisation, we found that the totality of CD11c+DC that captured the Ea-beads were only moDC and no cDC as shown by the phenotype of CD11c+ beads+DC (CD11b+CD8-F4/80-CD64+MAR-1+CCR2+, data not shown). Since we used a new model with particulate Ag, it was critical to validate our previous findings of the impact of CpG on Tfh-dependent B cell response with the beads. We thus immunised animals with OVA-beads in IFA or IFA with CpG and observed that the OVA-specific IgG response was enhanced after addition of CpG (data not shown). Similarly, using 1W1K-beads, we observed that addition of CpG to IFA increased 1W1K-specific Tfh cell numbers after immunisation (data not shown). We thus enumerated the number of moDC in dLN 2 days after immunisation with Ea-beads and found no statistical difference between IFA and IFA with CpG immunised animals (data not shown). Further, we found that the frequency of IL-6 producing CD11c+ beads+moDC was increased in animals immunised with IFA with CpG than those with IFA control animals (data not shown). Noticeably, the number of IL-6-producing CD11c+ beads+moDC was dependent on the dose of CpG added to IFA (FIG. 4). These results demonstrate that addition of soluble CpG to vaccine adjuvant enhances the frequency and number of IL-6-producing moDC in vivo in the dLN.

IL-6 Production in Response to CpG Promotes Tfh Cell Differentiation

To explore whether highest production of IL-6 by CpG-sensitized Ag-presenting DC play a role in the enhancing effect induced by CpG, we next treated mice on days −1 and +4 (relative to immunisation with 1W1K) with a mAb that blocks IL-6 signalling in vivo (anti-IL-6Rα) and examined the activated CD4+ T cells in the dLN (data not shown). This treatment had no effect on total CD4+ T cell activation in the dLN (data not shown). As expected, in isotype control-treated animals, we observed that 1W1K-specific Tfh cells were more numerous in animals immunised with IFA with CpG when compared to IFA immunised ones (FIG. 5A). In contrast, this boosting effect was suppressed in anti-IL-6Rα-treated animals (FIG. 5A). Finally, to directly document the role of IL-6 produced by Ag-presenting DC, C57Bl/6 recipients were lethally irradiated before reconstitution with 70% CD11c-DTR BM supplemented with 30% BM from IL-6K0 mice. The resulting BM chimeras were treated with DTx and were immunised with 1W1K in IFA or IFA with CpG and the 1W1K-specific Tfh cell response was monitored. We found that absence of IL-6 production in CD11c+DC resulted in the absence of Tfh differentiation enhancement due to CpG (FIG. 5B). These results collectively demonstrate that addition of soluble CpG to IFA directly increases the production of IL-6 by Ag-presenting DC that, in turn, enhances Tfh cell differentiation in vivo.

CD11b+Monocyte-Derived DC Promote Tfh Cell Responses

We then turned our attention to test which CD11b+DC subset, moDC and/or cDC, mediated the enhancing effect on Tfh cell differentiation in vivo after CpG addition. First, we depleted in vivo monocytes (CD11b+CD115+Ly6C+) using clodronate encapsulated in liposome (data not shown). The resulting animals were immunised with 1W1K or OVA in IFA or IFA with CpG. We found that the 1W1K-specific Tfh cell compartment and the OVA-specific IgG response were increased after CpG addition only in PBS control treated animals but not in clodronate ones (FIG. 6A). Moreover, we also found no increase of the Tfh compartment after IFA with CpG immunisation in mice in which monocyte recruitment is impaired (CX3CR1KO mice and CCR2KO chimeric mice) (FIG. 6B). One striking feature was that the 1W1K-specific Tfh compartment still developed and to the same level in IFA or IFA with CpG in animals depleted of monocytes or in which monocyte recruitment is impaired (FIGS. 6A and B). Altogether, this series of experiments demonstrated that cDC prime Tfh cells and that moDC orchestrate the enhancing effect driven by CpG on Tfh cell differentiation.

Addition of CpG to RIM Promotes Ag-Specific Tfh Cell Development in a Dose-Dependent Manner and Allows to Lower the Amount of Ag

We followed the 1W1K-specific CD4+ T cells with the corresponding pMHCII tetramer in the dLN after s.c. immunisation in RIBI complemented with CpG. At the peak of the effector CD4+ T cell response, we found that addition of CpG to RIBI, irrespective of the dose used, did not statistically change the frequency of CD4+ T cells in the dLN (FIG. 7A) nor the one of 1W1K-specific CD4+ T cells (FIG. 7B). Strikingly, we observed that addition of CpG to RIBI (or SAS) induced a dose-dependent increase of 1W1K-specific Tfh cells in the dLN (FIG. 7C). These results demonstrate that addition of soluble TLR9 agonist to RIBI enhances the pool of Ag-specific Tfh cells in vivo without affecting the overall magnitude of the Ag-specific CD4+ T cell compartment.

We next tested whether this increase of Tfh cell response could provide a way to decrease the amount of Ag to use in the emulsion used for vaccination. In this order, we immunized animals with different dose of 1W1K in RIBI complemented with 50 μg/mL of CpG. At the peak of the effector CD4+ T cell response, we found that decreasing Ag dose did not statistically change the frequency of CD4+ T cells in the dLN (FIG. 8A) nor the one of 1W1K-specific CD4+ T cells (FIG. 8B). Strikingly, we observed that even with decreasing dose of Ag, addition of CpG to RIBI (or SAS) still induced an increase of 1W1K-specific Tfh cells in the dLN except for the smallest 1W1K dose used (FIG. 8C). These results demonstrate that addition of soluble TLR9 agonist to RIBI enhances the pool of Ag-specific Tfh cells in vivo without affecting the overall magnitude of the Ag-specific CD4+ T cell compartment and could be used to lower the amount of Ag in the vaccine emulsion.

Addition of CpG to RIM Increases the Amount of Antigen-Presenting moDC

We demonstrated that cDC prime Tfh cells and that moDC drive the increase of Tfh cell differentiation. In order to test whether this was also true when CpG was added to RIBI we monitored antigen-presentation in the dLN 2 days after immunization. We found that addition of CpG to RIBI, irrespective of the CpG dose, had no effect on the total cell count number nor on the total number of DC (data not shown). Anyhow, while CpG addition had no effect on the Ag presentation by cDC (FIG. 9A), it increased in a dose dependent-manner the frequency of Ag-presenting moDC, which orchestrate the enhancing effect driven by CpG on Tfh cell differentiation.

Addition of CpG to Vaccine Adjuvant Promotes Better Protection to Pathogen Challenges

In this next series of experiments, we are testing how combination of adjuvant modifies the extent and longevity of the Ag-specific immunological memory and how they promote long-lasting protection depending on a pathogen infection. More precisely, we are testing how addition of CpG to RIBI modifies the extent and longevity of the Ag-specific immunological memory and how they promote long-lasting protection to pathogen infection. We immunize animals with either Hemagglutinin or attenuated Influenza virus in RIBI with or without CpG. 60 days after immunization, intranasal infection with lethal dose of the strain PR8 of Influenza virus is performed and survival as well as weight loss is monitored. Same is done after injection of tachizoites of toxoplasma gondii in mice challenged with SAG-1, a surface Ag of the parasite against which neutralising Ab are induced. Mice that are vaccinated with RIBI complemented with CpG are less susceptible to letal infection than the ones vaccinated with RIBI without CpG. Therefore, it shows that addition of CpG to RIBI induces a better protection to pathogen challenges.

### CONCLUSION

To be effective, protein vaccine must induce the development of a distinct lineage of CD4⁺ T cells named T Follicular Helper cells (Tfh), which regulate the differentiation of high-affinity memory B cells and long-lived plasma cells. We found that unexpectedly adjuvantation of vaccine adjuvant such as RIBI with CpG oligonucleotides, the TLR9 ligands validated for clinical use, promotes germinal centre reaction and enhances Ab response. This correlates with an increase of Tfh cells while the overall magnitude of the Ag-specific T cell response is unchanged. We comprehensively demonstrated that, in addition to the classical Tfh differentiation mediated by cDC, the promoting effect due to CpG was orchestrated in vivo by antigen presentation and IL-6 secretion by moDC as shown in their absence. Thus, while cDC initiate T cell responses; targeting moDC specifically enhances the Tfh program needed to regulate high-affinity B cell protection to pathogen challenges in vivo.

### REFERENCES

Throughout this application, various references describe the state of the art to which this invention pertains. The disclosures of these references are hereby incorporated by reference into the present disclosure.
Banchereau, J., and Steinman, R. M. (1998). Dendritic cells and the control of immunity. Nature 392, 245-252.
Crotty, S. (2011). Follicular Helper CD4 T Cells (T(FH)). Annu. Rev. Immunol. 29, 621-663.
Cucak, H., Yrlid, U., Reizis, B., Kalinke, U., and Johansson-Lindbom, B. (2009). Type I interferon signaling in dendritic cells stimulates the development of lymph-node-resident T follicular helper cells. Immunity 31, 491-501.
Fazilleau, N., Eisenbraun, M. D., Malherbe, L., Ebright, J. N., Pogue-Caley, R. R., McHeyzer-Williams, L. J., and McHeyzer-Williams, M. G. (2007). Lymphoid reservoirs of antigen-specific memory T helper cells. Nat Immunol 8, 753-761.
Fazilleau, N., McHeyzer-Williams, L. J., Rosen, H., and McHeyzer-Williams, M. G. (2009). The function of follicular helper T cells is regulated by the strength of T cell antigen receptor binding. Nat Immunol 10, 375-384
Hou, B., Saudan, P., Ott, G., Wheeler, M. L., Ji, M., Kuzmich, L., Lee, L. M., Coffman, R. L., Bachmann, M. F., and DeFranco, A. L. (2011). Selective utilization of Toll-like receptor and MyD88 signaling in B cells for enhancement of the antiviral germinal center response. Immunity 34, 375-384.
Janeway C A Jrl, Medzhitov R. Innate immune recognition. Annu Rev Immunol. 2002; 20:197-216. Epub 2001 Oct. 4.
Klinman, D. M. (2004). Immunotherapeutic uses of CpG oligodeoxynucleotides. Nat Rev Immunol 4, 249-258.
Krieg Arthur M. CPG motifs in bacterial DNA and their immune effects. Annu Rev. Immunol. 2002. 20:709-760.
Mata-Haro, V., Cekic, C., Martin, M., Chilton, P. M., Casella, C. R., and Mitchell, T. C. (2007). The vaccine adjuvant monophosphoryl lipid A as a TRIF-biased agonist of TLR4. Science 316, 1628-1632.
Pulendran, B., and Ahmed, R. (2011). Immunological mechanisms of vaccination. Nature immunology 131, 509-517.
Sapoznikov, A., Fischer, J. A., Zaft, T., Krauthgamer, R., Dzionek, A., and Jung, S. (2007). Organ-dependent in vivo priming of naive CD4+, but not CD8+, T cells by plasmacytoid dendritic cells. J Exp Med 204, 1923-1933.

1. An immunoadjuvant composition comprising:
a. at least one adjuvant and;
b. at least one MyD88-dependent pathway agonist.

2. An immunoadjuvant composition according to claim 1 wherein the adjuvant is selected from the group consisting of IFA, Alum, squalene and RIBI.

3. An immunoadjuvant composition according to claim 1 wherein the MyD88-dependent pathway agonist is selected from the group consisting of a TLR4 agonist, a TLR9 agonist and a mixture thereof.

4. An immunoadjuvant composition according to claim 3 wherein the TLR4 agonist is a lipopolysaccharide (LPS).

5. An immunoadjuvant composition according to claim 3 wherein the TLR9 agonist is a CpG nucleic acid (ODN).

6. An immunoadjuvant composition according to claim 5 wherein the TLR9 agonist is a CpG-B nucleic acid.

7. An immunoadjuvant composition according to claim 2 comprising IFA and LPS.

8. An immunoadjuvant composition according to claim 2 comprising IFA and CpG.

9. An immunoadjuvant composition according to claim 2 comprising IFA and CpG-B.

10. An immunoadjuvant composition according to claim 2 comprising RIBI and LPS.

11. An immunoadjuvant composition according to claim 2 comprising RIBI and CpG.

12. An immunoadjuvant composition according to claim 2 comprising RIBI and CpG-B.

13. An immunoadjuvant composition according to claim 1 comprising at least one adjuvant, at least one TLR4 agonist and at least one TLR9 agonist.

14. An immunoadjuvant composition according to claim 13 comprising the adjuvant IFA, the TLR4 agonist LPS and the TLR9 agonist CpG (ODN).

15. An immunoadjuvant composition according to claim 14 comprising the adjuvant IFA, the TLR4 agonist LPS and the TLR9 agonist CpG-B.

16. An immunoadjuvant composition according to claim 13 comprising the adjuvant ALUM, the TLR4 agonist LPS and the TLR9 agonist CpG.

17. An immunoadjuvant composition according to claim 13 comprising the adjuvant ALUM, the TLR4 agonist LPS and the TLR9 agonist CpG-B.

18. An immunoadjuvant composition according to claim 13 comprising the adjuvant RIBI, the TLR4 agonist LPS and the TLR9 agonist CpG.

19. An immunoadjuvant composition according to claim 13 comprising the adjuvant RIBI, the TLR4 agonist LPS and the TLR9 agonist CpG-B.

20. A method for inducing Tfh development and improving a humoral response in a subject in need thereof comprising
administering to the subject a pharmacologically effective amount of an immunoadjuvant composition comprising
a. at least one adjuvant and;
b. at least one MyD88-dependent pathway agonist.

21. A vaccine composition comprising an immunoadjuvant composition comprising
a. at least one adjuvant and;
b. at least one MyD88-dependent pathway agonist and
one or more antigens.
